(19) United States
(12) Patent Application Publication
(10) Pub. No.: US 2002/0035106 A1
(43) Pub. Date: Mar. 21, 2002

(54) Use of potassium channel agonists for reducing fat food consumption

(76) Inventors: John Bondo Hansen, Jyderup (DK); Christina Bjenning, Putnam Valley, NY (US)

Correspondence Address: Reza Green, Esq., Novo Nordisk of North America, Inc., Suite 6400, 405 Lexington Avenue, New York, NY, 10174-6401, US

(21) Appl. No.: 09/891,691

(22) Filed: Jun. 26, 2001

Related U.S. Application Data
(60) Provisional application No. 60/217,930, filed on Jul. 13, 2000.

(30) Foreign Application Priority Data
Jun. 26, 2000 (DK) PA 2000 00987

(51) Int. Cl. A61K 31/549

(52) U.S. Cl. 514/223.2; 514/224.2

(57) ABSTRACT

The present invention relates to the use of potassium channel agonists for reducing or lowering the consumption of fat food. The present invention also embraces the use of the compounds of general formulas (I) and (Ia) in reducing or lowering the intake of fat food and methods of using the compounds and their pharmaceutical compositions.

### CROSS-REFERENCE TO RELATED APPLICATIONS

This application claims priority under 35 U.S.C. 119 of Danish application PA 2000 00987 filed on Jun. 26, 2000, and U.S. provisional application No. 60/217,930 filed on Jul. 13, 2000, the contents of which are fully incorporated herein by reference.

### FIELD OF THE INVENTION

The present invention relates to the use of the compounds of general formulas (I) and (Ia) for reducing or lowering the consumption of fat food. The present invention also embraces the use of potassium channel agonists in reducing or lowering the intake of fat food and methods of using the compounds and their pharmaceutical compositions.

### BACKGROUND OF THE INVENTION

Our feeding habits have changed drastically over the last century. The western population have doubled the daily intake of kilocalories while assuming more sedentary lifestyle. Fat intake in particular has increased dramatically so that now up to 50% of the total kilocalories consumed are fat-derived. In parallel, the incidence of obesity has skyrocketed, and along with that the risk of morbidity from hypertension, dyslipidemia, type 2 diabetes, coronary heart disease, stroke, gallbladder disease, osteoarthritis, sleep apnea and respiratory problems, and endometrial-, breast-, prostate- and colon cancers. Higher body-weights are also associated with increases in all-cause mortality.

Feeding behaviour is governed by a redundant system that has numerous afferent inputs, evolution has selected our physiology and behaviour to favour over-consumption rather than under-consumption. Ingestion of fat generates reward and generates strong signals that suppress appetite, but of all the macronutrients, fat is the least effective at suppressing appetite.

In view of the accumulating evidence that the type and amount of fat in the diet influence the risk of obesity, hypertension, dyslipidemia, type 2 diabetes, coronary heart disease, stroke, gallbladder disease, osteoarthritis, sleep apnea and respiratory problems, and certain forms of cancers, public health campaigns throughout the world are attempting to modify fat intake patterns. The ‘population nutrient goals’ proposed by the WHO study group on diet, nutrition and prevention of non-communicable diseases consist of a lower limit of 15% of energy from fat and an upper interim limit of 30%, possibly to be reduced in the future.

Drugs that presently are available for the treatment of obesity do no specifically attempt to limit the urge to consume kilocalories derived from fat. Sibutramine® interferes with brain serotonergic signalling, classically connected to a.m. carbohydrate intake and Xenical® reduces the absorption of lipids from the gut.

Potassium channels play an important role in membrane potential. Among the different types of potassium channels are the ATP-sensitive (K_{ATP}-) channels, which are regulated by changes in the intracellular concentration of nucleotides. The K_{ATP}-channels have been found in cells from various tissues such as cardiac cells, pancreatic-cells, skeletal muscles, smooth muscles, central neurons, adipocytes and adenohypophysis cells. The channels have been associated with diverse cellular functions for example hormone secretion (insulin from pancreatic beta-cells, growth hormone and prolactin from adenohypophysis cells), vasodilation (in smooth muscle cells), cardiac action potential duration, neurotransmitter release in the central nervous system and lipid metabolism.

The K_{ATP}-channel exists as an octameric complex of the sufonylurea receptor (SUR) and the poreforming indwardly rectifying potassium channel (Kir) in a 4+4 stoichiometry. The activity of the channels is regulated by intracellular nucleotides and by different drugs. Whereas ATP and certain sulfonylureas are inhibitors (blockers), MgADP and potassium channel openers stimulate potassium currents. The genes for two closely related sulfonylurea receptors SUR1 and SUR2 have been cloned. Two different slice variants of SUR2, SUR2A and SUR2B have been reported. SUR1 combines with Kir6.2 to form the K_{ATP}-channels of pancreatic beta cells and neurones, whereas the cardiac type consists of SUR2A and Kir6.2 and the smooth muscle type of SUR2B and Kir6.1 or Kir6.2.

It has been shown that diazoxide (7-chloro-3-methyl-2H-1,2,4-benzothiadiazine 1,1-dioxide) and certain 3-(alkylamino)-4H-pyrido[4,3-e]-1,2,4-thiadiazine 1,1-dioxide derivatives inhibit insulin release by an activation of K_{ATP}-channels on pancreatic beta-cells (Pirotte B. et al., J. Med. Chem., 43, 1456-1466, (2000)). In obese Zucker rats, diazoxide has been shown to decrease insulin secretion and increase insulin receptor binding and consequently improve glucose tolerance and decrease weight gain (Alemzadeh R. et al. Endocrinol. 133, 705-712, 1993). In adipose tissue of Zucker rats, diazoxide has been found to down-regulate leptin and lipid metabolising enzymes (Standridge M et al. FASEB J. 14, 455-460, (2000). Upon 8 weeks treatment diazoxide had a significant antiobesity effect in hyperinsulinemic obese individuals (Alemzadeh et al. J. Clin. Endocrin. Metab., 83, 1911-1915, (1998)).

Intracerebroventricular administration of the potassium channel openers minoxidil, cromakalim and pinacidil, which activates K_{ATP}-channels of smooth muscle (SUR2B) without any effects on the K_{ATP}-channel of beta cells, have been reported to increase to intake of food by mice (Ghelardini C et al, Eur. J. Pharmacol, 329, 1-8, (1997)). In contrast, intraperitonal administration of cromakalim reduces the intake of food intake by rats (Del Prete, E, Pharmacol. Biochem. Behavior, 53, 839-842 (1996). Central administration of the blocker of K_{ATP}-channels, glibenclamide, reduce food intake by rats (Roane D. S. Pharmacol. Biochem. Behavior. 46, 205-207 (1993)).

K_{ATP}-channels are present on glucose responsive neurones in the brain. These K_{ATP}-channels may contain different portions of the SUR1, SUR2 and Kir6.2 subunits. Modulations of the K_{ATP}-channels through changes in the ATP/ADP ration give rise to changes in membrane potential and modulation of neurotransmitter release, which finally could influence food intake (Levin B, Am. J. Physiol. 276, R1223-R1231, (1991)).

Compounds that reduce snacking-behaviour in general and the preference for fat in particular, will have the potential to reduce all-cause mortality in general and in particular morbidity from hypertension, dyslipidemia, type 2 diabetes, coronary heart disease, stroke, gallbladder disease, osteoarthritis, sleep apnea and respiratory problems, and endometrial-, breast-, prostate- and colon cancers.

One object of the present invention is to provide compounds, which have a favourable impact on reducing the consumption of fat-derived calories.

### SUMMARY OF THE INVENTION

The present invention is based on the discovery that administration of compounds that are potassium channel openers have an effect on the intake of fat food fat, e.g. snack and can be used for reducing or lowering of the intake of fat food, such as e.g snacking.

Accordingly, the present invention provides the use potassium channel openers capable of reducing the consumption of fat food.

Further provided are the use of potassium channel openers, which are a β-cell selective potassium channel agonists.

The invention further provides the use of compounds of general formulas (I) and (Ia) for reducing or lowering the consumption of fat food consumption.

Further provided are pharmaceutical compositions comprising compounds that are potassium channel agonists and the compounds of the general formulas (I) and (Ia) or a salt thereof with a pharmaceutically acceptable acid or base.

The invention further provides a method for reducing or lowering fat food consumption, e.g. snacking.

### DETAILED DESCRIPTION OF THE INVENTION

Accordingly, the present invention provides the use of a compound of the general formula (I)

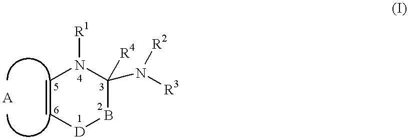

wherein

B represents >NR⁵ or >CR⁵R⁶, wherein R⁵ and R⁶ independently are hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen; or R⁵ and R⁴ together represent one of the bonds in a double bond between the atoms 2 and 3 of formula (I);

D represents —S(═O)₂— or —S(═O)—; or

D-B represents —S(═O)(R⁷)═N—

wherein R⁷ is C₁₋₆-alkyl; or aryl or heteroaryl optionally mono- or poly substituted with halogen, hydroxy, C₁₋₆-alkoxy, aryloxy, arylalkoxy, nitro, amino, C₁₋₆-monoalkyl- or dialkylamino, cyano, acyl, or C₁₋₆-alkoxycarbonyl;

R¹ is hydrogen; hydroxy; C₁₋₆-alkoxy; or C₁₋₆-alkyl, C₃₋₆-cycloalkyl, C₂₋₆- alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);

R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen;

R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₋₆-alkyl;

wherein R⁸ is hydrogen; C₃₋₆-cycloalkyl or (C₃₋₆-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;

X is O or S;

R⁹ is hydrogen; C₁₋₆-alkyl; C₂₋₆-alkenyl; C₃₋₆-cycloalkyl optionally mono- or poly substituted with C₁₋₆-alkyl, halogen, hydroxy or C₁₋₆-alkoxy; or

R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or

R³ is

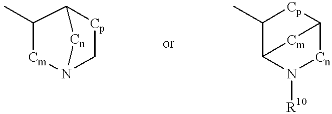

wherein n, m, p independently are 0,1,2,3 and R¹⁰ is hydrogen; hydroxy; C₁₋₆-alkoxy; C₃₋₆-cycloalkyl optionally mono- or poly substituted with C₁₋₆-alkyl, halogen, hydroxy or C₁₋₆-alkoxy; C₁₋₆-alkyl, C₂₋₆-alkenyl or C₂₋₆-alkynyl optionally mono- or poly substituted with halogen; or

R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;

A together with carbon atoms 5 and 6 of formula (I) represents a 5 or 6 membered heterocyclic system comprising one or more nitrogen-, oxygen- or sulfur atoms, the heterocyclic systems optionally being mono- or poly substituted with halogen; C₁₋₁₂-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl- methyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl- amino; C₁₋₆-monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)—C₁₋₆-alkyl the oxadiazolyl group optionally being substituted with C₁₋₆-alkyl or C₃₋₆-cycloalkyl; or a 5-6 membered nitrogen containing ring, optionally substituted with phenyl or C₁₋₆-alkyl; or

a salt thereof with a pharmaceutically acceptable acid or base, for the manufacture of a pharmaceutical composition for reducing or lowering the consumption of fat food.

Within its scope the invention includes all optical isomers of compounds of the present invention, some of which are optically active, and also their mixtures including racemic mixture thereof.

The scope of the invention also includes all tautomeric forms of the compounds of the present invention as well as metabolites or prodrugs.

A “metabolite” of a compound disclosed in this application is an active derivative of a compound disclosed herein which is produced when the compound is metabolized. Metabolites of compounds disclosed herein can be identified either by administration of a compound to a host and an analysis of blood samples from the host, or by incubation of compounds with hepatic cells in vitro and analysis of the incubant. A “prodrug” is a compound that either is converted into a compound disclosed in the application in vivo or has the same active metabolite as a compound disclosed in this application.

The salts include pharmaceutically acceptable acid addition salts, pharmaceutically acceptable metal salts or optionally alkylated ammonium salts, such as hydrochloric, hydrobromic, hydroiodic, phosphoric, sulfuric, trifluoroacetic, trichloroacetic, oxalic, maleic, pyruvic, malonic, succinic, citric, tartaric, fumaric, mandelic, benzoic, cinnamic, methanesulfonic, ethane sulfonic, picric and the like, and include acids related to the pharmaceutically acceptable salts listed in Journal of Pharmaceutical Science, 66, 2 (1977) and incorporated herein by reference, or lithium, sodium, potassium, magnesium and the like.

The term “fat food” as used herein refers to food for which more than 10% of the energy is derived from fat, e.g food containing from 10 kcal % fat, from 15 kcal % fat, 30 kcal % fat or from 45 kcal % fat.

The term “snacking” as used herein refers to an excessive consumption of food not related to hunger sensation.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy.

The term “C₁₋₆-alkylthio” as used herein, alone or in combination, refers to a straight or branched monovalent substituent comprising a lower alkyl group linked through a divalent sulfur atom having its free valence bond from the sulfur atom and having 1 to 6 carbon atoms e.g. methylthio, ethylthio, propylthio, butylthio, pentylthio.

The term “C₂₋₆-alkenyl” as used herein refers to an unsaturated hydrocarbon chain having 2-6 carbon atoms and one double bond such as e.g. vinyl, 1-propenyl, allyl, isopropenyl, n-butenyl, n-pentenyl and n-hexenyl.

The term C₃₋₆-cycloalkyl” as used herein refers to a radical of a saturated cyclic hydrocarbon with the indicated number of carbons such as cyclopropyl, cyclobutyl, cyclopentyl or cyclohexyl.

The term “C₂₋₆-alkynyl” as used herein refers to unsaturated hydrocarbons which contain triple bonds, such as e.g. —C≡CH, —C≡CCH₃, —CH₂C≡CH, —CH₂CH₂C≡CH, —CH(CH₃)C≡CH, and the like.

The term “C₁₋₆-alkoxy-C₁₋₆-alkyl” as used herein refers to a group of 2-12 carbon atoms interrupted by an O such as e.g. CH₂—O—CH₃, CH₂—O—CH₂—CH₃, CH₂—O—CH(CH₃)₂ and the like.

The term “halogen” means fluorine, chlorine, bromine or iodine.

The term “perhalomethyl” means trifluoromethyl, trichloromethyl, tribromomethyl or triiodomethyl.

The terms “C₁₋₆-alkyl”, “C₁₋₁₂-alkyl” and “C₁₋₈-alkyl” as used herein, alone or in combination, refers to a straight or branched, saturated hydrocarbon chain having the indicated number of carbon atoms such as e.g. methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-hexyl, 1,2-dimethylpropyl, 2,2-dimethylpropyl, 1,2,2-trimethylpropyl and the like. The term “C₁₋₈-alkyl” as used herein also includes secondary C₃₋₆-alkyl and tertiary C₄₋₆-alkyl.

The term “C₁₋₆-monoalkylamino” as used herein refers to an amino group wherein one of the hydrogen atoms is substituted with a straight or branched, saturated hydrocarbon chain having the indicated number of carbon atoms such as e.g. methylamino, ethylamino, propylamino, n-butylamino, sec-butylamino, isobutylamino, tert-butylamino, n-pentylamino, 2-methylbutylamino, n-hexylamino, 4-methylpentylamino, neopentylamino, n-hexylamino, 2,2-dimethylpropylamino and the like.

The term “C₁₋₆-dialkylamino” as used herein refers to an amino group wherein the two hydrogen atoms independently are substituted with a straight or branched, saturated hydrocarbon chain having the indicated number of carbon atoms; such as dimethylamino, N-ethyl-N-methylamino, diethylamino, dipropylamino, N-(n-butyl)-N-methylamino, di(n-pentyl)amino, and the like.

The term “acyl” as used herein refers to a monovalent substituent comprising a C₁₋₆-alkyl group linked through a carbonyl group; such as e.g. acetyl, propionyl, butyryl, isobutyryl, pivaloyl, valeryl, and the like.

The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, sec-butoxycarbonyl, tert-butoxycarbonyl, 3-methylbutoxycarbonyl, n-hexoxycarbonyl and the like.

The term “3-12 membered mono- or bicyclic system” as used herein refers to a monovalent substituent of formula —NR²R³ or —NR⁸R⁹ where R² and R³, or R⁸ and R⁹ together with the nitrogen atom form a 3-12 membered mono- or bicyclic system, in which one or more of the carbon atoms may be exchanged with nitrogen, oxygen or sulfur, such as 1-pyrrolidyl, piperidino, morpholino, thiomorpholino, 4-methylpiperazin-1-yl, 7-azabicyclo[2.2.1]heptan-7-yl, tropanyl and the like.

The term “3-6 membered saturated ring system” as used herein refers to a monovalent substituent comprising a monocyclic saturated system containing one or more hetero atoms selected from nitrogen, oxygen and sulfur and having 3-6 members and having its free valence from a carbon atom, e.g. 2-pyrrolidyl, 4-piperidyl, 3-morpholinyl, 1,4-dioxan-2-yl, 5-oxazolidinyl, 4-isoxazolidinyl or 2-thiomorpholinyl.

The term “bicycloalkyl” as used herein refers to a monovalent substituent comprising a bicyclic structure made of 6-12 carbon atoms such as e.g. 2-norbornyl, 7-norbornyl, 2-bicyclo[2.2.2]octyl and 9-bicyclo[3.3.1]nonanyl.

The term “aryl” as used herein refers to phenyl, 1-naphthyl or 2-naphthyl.

The term “heteroaryl” as used herein, alone or in combination, refers to a monovalent substituent comprising a 5-6 membered monocyclic aromatic system or a 9-10 membered bicyclic aromatic system containing one or more heteroatoms selected from nitrogen, oxygen and sulfur, e.g. pyrrole, imidazole, pyrazole, triazole, pyridine, pyrazine, pyrimidine, pyridazine, isothiazole, isoxazole, oxazole, oxadiazole, thiadiazole, quinoline, isoquinoline, quinazoline, quinoxaline, indole, benzimidazole, benzofuran, pteridine and purine.

The term “arylalkyl” as used herein refers to a straight or branched saturated carbon chain containing from 1 to 6 carbons substituted with an aromatic carbohydride; such as benzyl, phenethyl, 3-phenylpropyl, 1-naphtylmethyl, 2-(1-naphtyl)ethyl and the like.

The term “aryloxy” as used herein refers to phenoxy, 1-naphthyloxy or 2-naphthyloxy.

The term “arylalkoxy” as used herein refers to a C₁₋₆-alkoxy group substituted with an aromatic carbohydride, such as benzyloxy, phenethoxy, 3-phenylpropoxy, 1-naphthylmethoxy, 2-(1-naphtyl)ethoxy and the like.

The term “heteroarylalkyl” as used herein refers to a straight or branched saturated carbon chain containing from 1 to 6 carbons substituted with a heteroaryl group; such as (2-furyl)methyl, (3-furyl)methyl, (2-thienyl)methyl, (3-thienyl)methyl, (2-pyridyl)methyl, 1-methyl-1-(2-pyrimidyl)ethyl and the like.

The term “C₁₋₆-alkylsulfonyl” as used herein refers to a monovalent substituent comprising a C₁₋₆-alkyl group linked through a sulfonyl group such as e.g. methylsulfonyl, ethylsulfonyl, n-propylsulfonyl, isopropylsulfonyl, n-butylsulfonyl, sec-butylsulfonyl, isobutylsulfonyl, tert-butylsulfonyl, n-pentylsulfonyl, 2-methylbutylsulfonyl, 3-methylbutylsulfonyl, n-hexylsulfonyl, 4-methylpentylsulfonyl, neopentylsulfonyl, n-hexylsulfonyl and 2,2-dimethylpropyisulfonyl.

The term “C₁₋₆-monoalkylaminosulfonyl” as used herein refers to a monovalent substituent comprising a C₁₋₆-monoalkylamino group linked through a sulfonyl group such as e.g. methylaminosulfonyl, ethylaminosulfonyl, n-propylaminosulfonyl, isopropylaminosulfonyl, n-butylaminosulfonyl, sec-butylaminosulfonyl, isobutylaminosulfonyl, tert-butylaminosulfonyl, n-pentylaminosulfonyl, 2-methylbutylaminosulfonyl, 3-methylbutylaminosulfonyl, n-hexylaminosulfonyl, 4-methylpentylaminosulfonyl, neopentylaminosulfonyl, n-hexylaminosulfonyl and 2,2-dimethylpropylaminosulfonyl.

The term “C₁₋₆-dialkylaminosulfonyl” as used herein refers to a monovalent substituent comprising a C₁₋₆-dialkylamino group linked through a sulfonyl group such as dimethylaminosulfonyl, N-ethyl-N-methylaminosulfonyl, diethylaminosulfonyl, dipropylaminosulfonyl, N-(n-butyl)-N-methylaminosulfonyl, di(n-pentyl)aminosulfonyl, and the like.

The term “C₁₋₆-alkylsulfinyl” as used herein refers to a monovalent substituent comprising a straight or branched C₁₋₆-alkyl group linked through a sulfinyl group (—S(═O)—); such as e.g. methylsulfinyl, ethylsulfinyl, isopropylsulfinyl, butylsulfinyl, pentylsulfinyl, and the like.

The term “C₁₋₆-alkylcarbonylamino” as used herein refers to an amino group wherein one of the hydrogen atoms is substituted with an acyl group, such as e.g. acetamido, propionamido, isopropylcarbonylamino, and the like.

The term “(C₃₋₆-cycloalkyl)C₁₋₆-alkyl” as used herein, alone or in combination, refers to a straight or branched, saturated hydrocarbon chain having 1 to 6 carbon atoms and being monosubstituted with a C₃₋₆-cycloalkyl group, the cycloalkyl group optionally being mono- or polysubstituted with C₁₋₆alkyl, halogen, hydroxy or C₁₋₆-alkoxy; such as e.g. cyclopropylmethyl, (1-methylcyclopropyl)methyl, 1-(cyclopropyl)ethyl, cyclopentylmethyl, cyclohexylmethyl, and the like.

The term “arylthio” as used herein, alone or in combination, refers to an aryl group linked through a divalent sulfur atom having its free valence bond from the sulfur atom, the aryl group optionally being mono- or polysubstituted with C₁₋₆-alkyl, halogen, hydroxy or C₁₋₆alkoxy; e.g. phenylthio, (4-methylphenyl)- thio, (2-chlorophenyl) thio, and the like.

The term “arylsulfinyl” as used herein refers to an aryl group linked through a sulfinyl group (—S(═O)—), the aryl group optionally being mono- or polysubstituted with C₁₋₆-alkyl, halogen, hydroxy or C₁₋₆-alkoxy; such as e.g. phenylsulfinyl, (4-chlorophenyl)sulfinyl, and the like.

The term “arylsulfonyl” as used herein refers to an aryl group linked through a sulfonyl group, the aryl group optionally being mono- or polysubstituted with C₁₋₆-alkyl, halogen, hydroxy or C₁₋₆-alkoxy; such as e.g. phenylsulfonyl, tosyl, and the like.

The term “C₁₋₆-monoalkylaminocarbonyl” as used herein refers to a monovalent substituent comprising a C₁₋₆-monoalkylamino group linked through a carbonyl group such as e.g. methylaminocarbonyl, ethylaminocarbonyl, n-propylaminocarbonyl, isopropylaminocarbonyl, n-butylaminocarbonyl, sec-butylaminocarbonyl, isobutylaminocarbonyl, tert-butylaminocarbonyl, n-pentylaminocarbonyl, 2-methylbutylaminocarbonyl, 3-methylbutylaminocarbonyl, n-hexylaminocarbonyl, 4-methylpentylaminocarbonyl, neopentylaminocarbonyl, n-hexylaminocarbonyl and 2-2-dimethylpropylaminocarbonyl.

The term “C₁₋₆-dialkylaminocarbonyl” as used herein refers to a monovalent substituent comprising a C₁₋₆-dialkylamino group linked through a carbonyl group such as dimethylaminocarbonyl, N-ethyl-N-methylaminocarbonyl, diethylaminocarbonyl, dipropylaminocarbonyl, N-(n-butyl)-N-methylaminocarbonyl, di(n-pentyl)aminocarbonyl, and the like.

The term “C₁₋₆-monoalkylaminocarbonylamino” as used herein refers to an amino group wherin one of the hydrogen atoms is substituted with a C₁₋₆-monoalkylaminocarbonyl group, e.g. methylaminocarbonylamino, ethylamino-carbonylamino, n-propylaminocarbonylamino, isopropylaminocarbonylamino, n-butylaminocarbonylamino, sec-butylaminocarbonylamino, isobutylaminocarbonylamino, tert-butylaminocarbonylamino, and 2-methylbutylaminocarbonylamino.

The term “C₁₋₆-dialkylaminocarbonylamino” as used herein refers to an amino group wherein one of the hydrogen atoms is substituted with a C₁₋₆-dialkylaminocarbonyl group, such as dimethylaminocarbonylamino, N-ethyl-N-methylaminocarbonylamino, diethylaminocarbonylamino, dipropylaminocarbonylamino, N-(n-butyl)-N-methylaminocarbonylamino, di(n-pentyl)aminocarbonylamino, and the like.

The term “5- or 6-membered heterocyclic system” as used herein refers to: a monocyclic unsaturated or saturated system containing one, two or three hetero atoms selected from nitrogen, oxygen and sulfur and having 5 members, e.g. pyrrole, furan, thiophene, pyrroline, dihydrofuran, dihydrothiophene, imidazole, imidazoline, pyrazole, pyrazoline, oxazole, thiazole, isoxazole, isothiazole, 1,2,3-oxadiazole, furazan, 1,2,3-triazole, 1,2,3-thiadiazole or 2,1,3-thiadiazole; an aromatic monocyclic system containing one or more nitrogen atoms and having 6 members, e.g. pyridine, pyrazine, pyrimidine, pyridazine, 1,2,4-triazine, 1,2,3-triazine or tetrazine; a non-aromatic monocyclic system containing one or more hetero atoms selected from nitrogen, oxygen and sulfur and having 6 members, e.g. pyran, thiopyran, piperidine, dioxane, oxazine, isoxazine, dithiane, oxathine, thiazine, piperazine, thiadiazine, dithiazine or oxadiazine.

The term “5- or 6-membered nitrogen containing ring” as used herein refers to a monovalent substituent comprising a monocyclic unsaturated or saturated system containing one or more nitrogen atoms and having 5 or 6 members, e.g. pyrrolidinyl, pyrrolinyl, imidazolidinyl, pyrazolidinyl, pyrazolinyl, piperidyl, piperazinyl, pyrrolyl, 2H-pyrrolyl, imidazolyl, pyrazolyl, triazolyl, pyridyl, pyrazinyl, pyrimidinyl, pyridazinyl, morpholino, thiomorpholino, isothiazolyl, isoxazolyl, oxazolyl, oxadiazolyl, thiadiazolyl, 1,3-dioxolanyl and 1,4-dioxolanyl.

The term “4- to 12-membered bicyclic or tricyclic carbocyclic system” as used herein refers to a a monovalent substituent comprising a bicyclic or a tricyclic structure made of 4-12 carbon atoms such as e.g. bicyclo[2.1.1]hexane, bicyclo[2.2.1]heptane, bicyclo[2.2.2]octane, octahydrovpentalene, bicyclo[2.2.0]hexane, adamantane, noradamantane or tricyclo-(4.3.1.1(3,8))undecane.

In one embodiment of the invention B of formula (I) is >NR⁵ and R⁵ and R⁴ together represent one of the bonds in a double bond between the atoms 2 and 3 of formula (I).

In another embodiment of the invention D is —S(═O)₂—.

In another embodiment of the invention R² is hydrogen or C₁₋₆-alkyl.

In another embodiment of the invention R³ is R⁸, —OR⁸, NR⁸R⁹ or aryl, the aryl groups optionally being substituted with C₁₋₆-alkyl; wherein R⁸ is hydrogen; C₃₋₆-cycloalkyl; (C₃₋₆-cycloalkyl)C₁₋₆-alkyl; a 3-6 membered saturated ring system comprising one, two or three nitrogen-, oxygen- or sulfur atoms; or straight or branched C₁₋₁₈-alkyl optionally substituted with halogen, hydroxy, C₁₋₆-alkoxy, C₁₋₆-alkylthio, C₃₋₆-cycloalkyl or aryl, R⁹ is hydrogen, C₁₋₆-alkyl or C₃₋₆-cycloalkyl; or R⁸ and R⁹ together with the nitrogen atom form a 4-6 membered ring.

In another embodiment of the invention wherein R³ is secondary C₃₋₆-alkyl, tertiary C₄₋₆-alkyl, C₃₋₆-cycloalkyl or (C₃₋₆-cycloalkyl)methyl.

In another embodiment of the invention A together with carbon atoms 5 and 6 of formula (I) forms a 5 membered heterocyclic system containing one hetero atom selected from nitrogen and sulfur, the heterocyclic system optionally being mono- or disubstituted with halogen; C₁₋₁₂-alkyl; C₃₋₆-cycloalkyl; cyano; cyanomethyl; perhalomethyl; sulfamoyl; C₁₋₆-alkylthio; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl or a 5-6 membered nitrogen containing ring, optionally substituted with phenyl or C₁₋₆-alkyl.

In another embodiment of the invention A together with carbon atoms 5 and 6 of formula (I) forms a 5 membered heterocyclic system containing two hetero atoms selected from nitrogen, oxygen and sulfur, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

In another embodiment of the invention A together with carbon atoms 5 and 6 of formula (I) forms a 6 membered aromatic heterocyclic system containing one, two or three nitrogen atoms, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylthio; C₁₋₆alkylsulfonyl; C₁₋₆-alkylsulfinyl; arylthio, arylsulfinyl, arylsulfonyl, the aryll group optionally being mono- or poly-substituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl: aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

Examples of such specific compounds of formula (I) to be used according to this invention are: 6-Chloro-3-(1,2-dimethylpropyl)amino-4H-thieno[3,2-e]-1,2,4-thiadiazine 1,1-dioxide; 6-Chloro-3-ethylamino-4H-thieno[3,2-e]-1,2,4-thiadiazine 1,1-dioxide; 6-Chloro-3-isopropylamino-4H-thieno[3,2-e]-1,2,4-thiadiazine 1,1-dioxide; (R)-6-Chloro-3-(1 -phenylethyl)amino-4H-thieno[3,2-e]-1,2,4-thiadiazine 1,1-dioxide; 3-Allylamino-6-chloro-4H-thieno[3,2-e]-1,2,4-thiadiazine 1,1-dioxide; 6-Chloro-3-cyclopropylamino-4H-thieno[3,2-e]-1,2,4-thiadiazine 1,1-dioxide; 6-Chloro-3-hexylamino-4H-thieno[3,2-e]-1,2,4-thiadiazine 1,1-dioxide; 6-Chloro-3-tetradecylamino-4H-thieno[3,2-e]-1,2,4-thiadiazine 1,1-dioxide; 6-Chloro-3-methylamino-4H-thieno[3,2,e]-1,2,4-thiadiazine 1,1-dioxide; 3-Benzylamino-6chloro-4H-thieno[3,2,e]-1,2,4-thiadiazine 1,1-dioxide; 6-Chloro-3-octylamino-4H-thieno[3,2-e]-1,2,4-thiadiazine 1,1-dioxide; 6-Chloro-3-isobutylamino4H-thieno[3,2-e]-1,2,4-thiadiazine 1,1-dioxide; 6-Chloro-3-(4-phenylbutyl)amino-4H-thieno[3,2-e]-1,2,4-thiadiazine 1,1-dioxide; 6-Chloro-3-(1,5-dimethylhexyl)amino-4H-thieno[3,2-e]-1,2,4-thiadiazine 1,1-dioxide; 6-Chloro-3-propylamino-4H-thieno[3,2-e]-1,2,4-thiadiazine 1,1-dioxide; (R)-6-Chloro-3-(2-hydroxy-1-methylethyl)amino-4H-thieno[3,2-e]-1,2,4-thiadiazine 1,1-dioxide; (S)-6-Chloro-3-(2-hydroxy-1-methylethyl)amino-4H-thieno[3,2-e]-1,2,4-thiadiazine 1,1-dioxide; (R)-3-sec-Butylamino-6-chloro-4H-thieno[3,2-e]-1,2,4-thiadiazine 1,1-dioxide; 3-Butylamino-6-chloro-4H-thieno[3,2-e]-1,2,4-thiadiazine 1,1-dioxide; 3-Isopropylamino-7-methyl-4,7-dihydropyrazolo[4,3-e][1,2,4]thiadiazine 1,1-dioxide.

Another example of a specific compound of formula (I) to be used according to this invention is 6-Chloro-3-isopropylamino-4H-thieno[3,2-e]-1,2,4-thiadiazine 1,1-dioxide.

Other examples of specific compounds of formula (I) to be used according to this invention are: 3-Hydrazino-4H-pyrido[4,3-e]-1,2,4-thiadiazine 1,1-dioxide; 3-Benzylamino-4H-pyrido[4,3-e]-1,2,4-thiadiazine 1,1-dioxide; 3-(R)-(1-Phenylethylamino)-4H-pyrido[4,3-e]-1,2,4-thiadiazine 1,1-dioxide; 3-(S)-(1-Phenylethylamino)-4H-pyrido[4,3-e]-1,2,4-thiadiazine 1,1-dioxide; 3-Benzylamino-7-chloro4H-pyrido[2,3-e]-1,2,4-thiadiazine 1,1-dioxide; 7-Chloro-3-(R)-(1-phenylethylamino)-4H-pyrido[2,3-e]-1,2,4-thiadiazine 1,1-dioxide; 7-Chloro-3-(S)-(1′-phenylethylamino)4H-pyrido[2,3-e]-1,2,4-thiadiazine 1,1-dioxide; 3-Benzylamino-4H-pyrido[2,3-e]-1,2,4-thiadiazine 1,1-dioxide; 3-(R)-( 1 -Phenylethylamino)-4H-pyrido[2,3-e]-1,2,4-thiadiazine 1,1-dioxide; 3-(S)-(1-Phenylethylamino)-4H-pyrido[2,3-e]-1,2,4-thiadiazine 1,1-dioxide; 3-(Hexylamino)-4H- pyrido[4,3-e]-1,2,4-thiadiazine 1,1-dioxide; 7-Chloro-3-hexylamino-4H- pyrido[2,3-e]-1,2,4-thiadiazine 1,1-dioxide; 3-Octylamino-4H- pyrido[4,3-e]-1,2,4-thiadiazine 1,1-dioxide; 7-Chloro-3-octylamino-4H-pyrido [2,3-e]-1,2,4-thiadiazine 1,1-dioxide; 3-Allylamino-4H- pyrido[4,3-e]-1,2,4-thiadiazine 1,1-dioxide; 3-Allylamino-7-chloro-4H-pyrido[2,3-e]-1,2,4-thiadiazine 1,1-dioxide; 7-Chloro-3-(2-methoxy-1-methylethyl)amino-4H-pyrido[2,3-e]-1,2,4-thiadiazine 1,1-dioxide; 3-(2-Methoxy-1-methylethyl)amino-4H-pyrido[4,3-e]-1,2,4-thiadiazine 1,1-dioxide; 3-(2-Hydroxy-1-methylethyl)amino-4H-pyrido[4,3-e]-1,2,4-thiadiazine 1,1-dioxide; 3-Benzylamino-2-methyl-2H-pyrido[4,3-e]-1,2,4-thiadiazine 1,1-dioxide; 2-Isopropylamino-3,3-dimethoxy-3H-pyrido [2,3-b][1,4]thiazine 4,4-dioxide.

Other examples of specific compounds of formula (I) to be used according to this invention are: 7-Cyano-3-isopropylamino-6-methyl-4H-thieno[2,3-e]-1,2,4-thiadiazine 1,1-dioxide; 7-Cyano-6-methyl-3-propylamino-4H-thieno[2,3-e]-1,2,4-thiadiazine 1,1-dioxide; 6-Chloro-3-isopentylamino-4H-thieno[3,2-e]-1,2,4-thiadiazine 1,1-dioxide; 6-Chloro-3-(1 -methyl-heptyl)amino-4H-thieno[3,2-e]-1,2,4-thiadiazine 1,1-dioxide; 6-Chloro-3-(1-ethylpentyl)amino-4H-thieno[3,2-e]-1,2,4-thiadiazine 1,1-dioxide; 6-Chloro-3-(2-methylbutyl)amino-4H-thieno[3,2-e]-1,2,4-thiadiazine 1,1-dioxide; 6-Chloro-3-(1-methylhexyl)amino-4H-thieno[3,2-e]-1,2,4-thiadiazine 1,1-dioxide; 6-Chloro-3-cyclopentylamino-4H-thieno[3,2-e]-1,2,4-thiadiazine 1,1-dioxide; 6-Chloro-3-cyclohexylmethylamino-4H-thieno[3,2-e]-1,2,4-thiadiazine 1,1-dioxide; Ethyl 3-(6-chloro-1,4-dihydro-1,1-dioxothieno[3,2-e]-1λ⁶,2,4-thiadiazin-3-ylamino)-butanoate; 3-(6-Chloro-1,4-dihydro-1,1-dioxothieno[3,2-e]-1λ⁶,2,4-thiadiazin-3-ylamino)butanoic acid; 6-Chloro-3-(3-hydroxy-1 -methylpropyl)amino-4H-thieno[3,2-e]-1,2,4-thiadiazine 1,1-dioxide; (R)-6-Chloro-3-(1 -phenylethyl)amino-4H-thieno[3,2-e]-1,2,4-thiadiazine 1,1-dioxide; (S)-3-sec-Butylamino-6-chloro-4H-thieno[3,2-e]-1,2,4-thiadiazine 1,1-dioxide; 6-Chloro-3-isopropylamino-4H-thieno[2,3-e]-1,2,4-thiadiazine 1,1-dioxide; 6-Chloro-3-cyclopentylamino-4H-thieno[2,3-e]-1,2,4-thiadiazine 1,1-dioxide; 6-Bromo-3-isopropylamino4H-thieno[3,2-e]-1,2,4-thiadiazine 1,1-dioxide; 3-Isopropylamino-4H-thieno[3,2-e]-1,2,4-thiadiazine 1,1-dioxide; 6-Fluoro-3-isopropylamino-4H-thieno[3,2-e]-1,2,4-thiadiazine 1,1-dioxide; 3-Cyclobutylamino-5,6-dimethyl-4H-thieno[3,2-e]-1,2,4-thiadiazine 1,1-dioxide; 3-Cyclopentylamino-5,6-dimethyl-4H-thieno[3,2-e]-1 ,2,4-thiadiazine 1,1-dioxide; 3-Isopropylamino-6,7-dimethyl-4H-thieno[2,3-e]-1,2,4-thiadiazine 1,1-dioxide; 3-Cyclobutylamino-6,7-dimethyl-4H-thieno[2,3-e]-1,2,4-thiadiazine 1,1-dioxide; 3-Cyclopentylamino-6,7-dimethyl-4H-thieno[2,3-e]-1,2,4-thiadiazine, 1 -dioxide; 5-Choro-3-isopropylamino-4H-thieno[3,2-e]-1,2,4thiadiazine 1,1-dioxide; 5-Chloro-3-propylamino-4H-thieno[3,2-e]-1,2,4-thiadiazine 1,1-dioxide; 5-Chloro-3-cyclopentylamino-4H-thieno[3,2-e]-1,2,4-thiadiazine 1,1-dioxide; 5-Chloro-6-methyl-3-isopropylamino-4H-thieno[3,2-e]-1,2,4-thiadiazine 1,1-dioxide; 6-chloro-3-isopropylamino-5-methyl-4H-thieno[3,2-e]-1 ,2,4-thiadiazine 1,1-dioxide; 6-chloro-3-cyclopentylamino-5-methyl-4H-thieno[3,2-e]-1,2,4-thiadiazine 1,1-dioxide; 6-Fluoro-3-propylamino-4H-thieno[3,2-e]-1,2,4-thiadiazine 1,1-dioxide; 6-Fluoro-3-cyclopentylamino-4H-thieno[3,2-e]-1,2,4-thiadiazine 1,1-dioxide; 5-Fluoro-3-propylamino-4H-thieno[3,2-e]-1,2,4-thiadiazine 1,1-dioxide; 5-Fluoro-3-isopropylamino-4H-thieno[3,2-e]-1,2,4-thiadiazine 1,1-dioxide; 3-isopropylamino-7-methyl-4H-thieno[2,3-e]-1,2,4-thiadiazine 1,1-dioxide; 6-Chloro-3-cyclobutylamino-4H-thieno[3,2-e]-1,2,4-thiadiazine 1,1-dioxide; 6-Chloro-3-(2-hydroxyethyl)amino-4H-thieno[3,2-e]-1,2,4-thiadiazine 1,1-dioxide; (±)-3-exo-Bicyclo[2.2.1 ]hept-2-ylamino-6-chloro-4H-thieno[3,2-e]-1,2,4-thiadiazine 1,1-dioxide; (R)-6-Chloro-3-(2-hydroxypropyl)amino-4H-thieno[3,2-e]-1,2,4-thiadiazine 1,1-dioxide; 6-Bromo-3-isopropylamino-4H-thieno[3,2-e]-1,2,4-thiadiazine 1,1-dioxide; 5,6-Dibromo-3-isopropylamino-4H-thieno[3,2-e]-1,2,4-thiadiazine 1,1-dioxide; 6-Chloro-3-cyclohexylamino-4H-thieno[3,2-e]-1,2,4-thiadiazine 1,1-dioxide;6-Chloro-3-(furan-2-ylmethyl)amino-4H-thieno[3,2-e]-1,2,4-thiadiazine 1,1-dioxide; 6-Chloro-3-(1-ethylpropyl)amino-4H-thieno[3,2-e]-1,2,4-thiadiazine 1,1-dioxide; 6-Bromo-3-cyclopentylamino-4H-thieno[3,2-e]-1,2,4-thiadiazine 1,1-dioxide; 6-Chloro-3-(2-methylallyl)amino-4H-thieno[3,2-e]-1,2,4-thiadiazine 1,1-dioxide; 6-Cyano-3-isopropylamino-4H-thieno[3,2-e]-1,2,4-thiadiazine 1,1-dioxide.

In another embodiment of the invention the general formula (I) is selected from

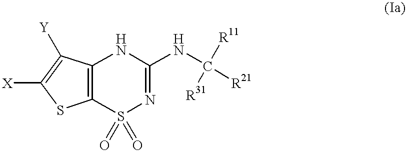

wherein

X and Y independently are hydrogen, halogen, perhalomethyl, C₁₋₆-alkyl or C₁₋₆-alkoxy;

R¹¹, R²¹ and R³¹ independently are C₁₋₆-alkyl, C₂₋₆-alkenyl, C₂₋₆-alkynyl, C₃₋₆-cycloalkyl, carboxy, C₁₋₆-alkoxycarbonyl or aryl, all of which are optionally being mono- or polysubstituted with halogen, hydroxy, oxo, or aryl; or

R¹¹ is as defined above and R²¹—C—R³¹ form a C₃₋₆-cycloalkyl group, optionally being mono- or polysubstituted with C₁₋₆-alkyl, perhalomethyl, halogen, hydroxy or aryl; or

—CR¹¹R²¹R³¹ form a 4- to 12-membered bicyclic or tricyclic carbocyclic system, optionally being mono- or polysubstituted with C, ₆-alkyl, perhalomethyl, halogen, hydroxy or aryl; or a salt thereof with a pharmaceutically acceptable acid or base including all optical isomers of compounds of formula (Ia).

In another embodiment of the invention in formula (Ia) X is halogen and Y is hydrogen.

In another embodiment of the invention in formula (Ia), X is chloro.

In another embodiment of the invention in formula (Ia), R¹¹, R²¹ and R³¹ all are C₁₋₆-alkyl.

In another embodiment of the invention in formula (Ia), R¹¹ is methyl.

In another embodiment of the invention in formula (Ia), R²¹—C—R³¹ forms a C₃₋₆-cycloalkyl group.

In another embodiment of the invention in formula (Ia), —CR¹¹R²¹R³¹ forms a tricyclic carbocyclic system.

Examples of such specific compounds of formula (Ia) to be used according to this invention are: 3-tert-Butylamino-6-chloro-4H-thieno[3,2-e]-1,2,4-thiadiazine 1,1-dioxide; 6-Chloro-3-(1,1-dimethylpropylamino)-4H-thieno[3,2-e]-1,2,4-thiadiazine 1,1-dioxide; 6-Chloro-3-(1-methylcyclopropyl)amino-4H-thieno[3,2-e]-1,2,4-thiadiazine 1,1-dioxide; 6-Chloro-3-(2-hydroxy-1,1-dimethylethylamino)-4H-thieno[3,2-e]-1,2,4-thiadiazine 1,1-dioxide; 6-Chloro-3-(1,1,3,3-tetramethylbutylamino)-4H-thieno[3,2-e]-1,2,4-thiadiazine 1,1-dioxide; 3-(1-Adamantyl)amino-6-chloro-4H-thieno[3,2-e]-1,2,4-thiadiazine 1,1-dioxide; 1-(6-Chloro-1,4-dihydro-1,1-dioxo-thieno[3,2-e]-1λ⁶,2,4-thiadiazin-3-ylamino)-cyclopropanecarboxylic acid ethyl ester; 6-Chloro-3-(1-methyl-1-phenylethyl)amino-4H-thieno[3,2-e]-1,2,4-thiadiazine 1,1-dioxide; 6-Chloro-3-(1-hydroxymethylcyclopentyl)amino-4H-thieno[3,2-e]-1,2,4-thiadiazine 1,1-dioxide; 1-(6-Chloro-1,4-dihydro-1,1-dioxo-thieno[3,2-e]-1λ⁶,2,4-thiadiazin-3-ylamino)-cyclopropanecarboxylic acid; 6-Chloro-3-(1-methylcyclobutyl)amino-4H-thieno[3,2-e]-1,2,4-thiadiazine 1,1-dioxide; 6-Chloro-3-(1-methylcyclohexyl)amino-4H-thieno[3,2-e]-1,2,4-thiadiazine 1,1-dioxide; 6-Chloro-3-(1-methylcyclopentyl)amino-4H-thieno[3,2-e]-1,2,4-thiadiazine 1,1-dioxide; 6-Chloro-3-(1-ethylcyclobutyl)amino-4H-thieno[3,2-e]-1,2,4-thiadiazine 1,1-dioxide.

Another example of a specific compound of formula (Ia) to be used according to this invention is 6-Chloro-3-(1-methylcyclopropyl)amino-4H-thieno[3,2-e]-1,2,4-thiadiazine 1,1-dioxide.

In another embodiment, the present invention relates to the use of compounds, which are potassium channel agonists for the manufacture of a pharmaceutical composition for reducing or lowering the consumption of fat food, e.g. snacking.

An example of such potassium channel agonist is diazoxide (7-chloro-3-methyl-2H-1,2,4-benzothiadiazine 1,1-dioxide).

Other examples of such potassium channel agonists are compounds, which activate K_{ATP}-channels of the beta cell type (SUR1/Kir6.2).

In another embodiment, the present invention relates to the use potassium channel agonists, which are a β-cell selective potassium channel agonists.

In another embodiment, the compounds of the present invention can be used for reducing or lowering the consumption of fat food containing from 10 kcal % fat.

In another embodiment, the compounds of the present invention can be used for reducing or lowering the consumption of fat food containing from 15 kcal % fat.

In another embodiment, the compounds of the present invention can be used for reducing or lowering the consumption of fat food containing from 30 kcal % fat.

In another embodiment, the compounds of the present invention can be used for reducing or lowering the consumption of fat food containing from 45 kcal % fat.

In another embodiment, the compounds of the present invention can be used for reducing or lowering the consumption of fat food, which is related to snacking.

In another embodiment, the compounds of the present invention can be used in methods for reducing lowering the consumption of fat food comprising administering to a subject in need thereof an effective amount of a compound of the present invention.

In another embodiment, the compounds of the present invention can be used to reduce all-cause mortality in general and in particular morbidity from hypertension, dyslipidemia, type 2 diabetes, coronary heart disease, stroke, gallbladder disease, osteoarthritis, sleep apnea and respiratory problems, and endometrial-, breast-, prostate- and colon cancers.

In yet another embodiment, the potassium channel compounds of the present invention may be used alone or in combination with one or more other pharmacologically active compounds, e.g. compounds that specifically reduce carbohydrate cravings or compounds that prevent the absorption of lipids from the food into the gastrointestinal canal.

In addition the compounds of the present invention may be used in combination with compounds that are used for the treatment of type 2 diabetes, obesitas or hypertension.

Potassium channel agonists can readily be determined by those skilled in the art. Methods therefore has been described in e.g. WO 97126264, WO 97126265, WO 99/03861, WO 00/37474 and recently reviewed: McClenaghan: Diabetes, Obesitas and Metabolism, 1, 137-150, (1999); Yokoshiki: Am. J. Physiol. . 274. C25-C37, (1998); Aguliar-Bryan: Endocrine Reviews, 20, 101-135, (1999).

The compounds of formula (I) and (Ia) of the present invention may be prepared by using the methods taught in e.g. WO 97/26264, WO 97/26265, WO 99/03861, WO 00/37474, which are hereby incorporated by reference.

PHARMACEUTICAL COMPOSITIONS The present invention also relates to pharmaceutical compositions comprising, as an active ingredient, at least one of the compounds of the present invention or a pharmaceutically acceptable salt thereof and, usually, such compositions also contain a pharmaceutically acceptable carrier or diluent.

Pharmaceutical compositions comprising a compound of the present invention may be prepared by conventional techniques, e.g. as described in Remington: The Science and Practise of Pharmacy, 19^{th} Ed., 1995. The compositions may appear in conventional forms, for example capsules, tablets, aerosols, solutions or suspensions.

Typical compositions include a compound of the present invention or a pharmaceutically acceptable acid addition salt thereof, associated with a pharmaceutically acceptable excipient which may be a carrier or a diluent or be diluted by a carrier, or enclosed within a carrier which can be in form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier, which may be in the form of a ampoule, capsule, sachet, paper, or other container. When the carrier serves as a diluent, it may be solid, semi-solid, or liquid material, which acts as a vehicle, excipient, or medium for the active compound. The active compound can be adsorbed on a granular solid container for example in a sachet. Some examples of suitable carriers are water, salt solutions, alcohols, polyethylene glycols, polyhydroxyethoxylated castor oil, syrup, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. The formulations may also include wetting agents, emulsifying and suspending agents, preserving agents, sweetening agents or flavouring agents.

The pharmaceutical preparations can be sterilized and mixed, if desired, with auxiliary agents, emulsifiers, salt for influencing osmotic pressure, buffers and/or coloring substances and the like, which do not deleteriously react with the active compounds.

The route of administration may be any route, which effectively transports the active compound to the appropriate or desired site of action, such as oral, nasal, pulmonary, transdermal or parenteral e.g. rectal, depot, subcutaneous, intramuscular or intranasal, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound of the present invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

The compounds of the invention may be administered to a mammal, especially a human, in need of such reducing or lowering of the intake of fat food. Such mammals include also animals, both domestic animals, e.g. household pets, and non-domestic animals such as wildlife.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, in an effective amount.

Pharmaceutical compositions containing a compound according to the invention may be administered one or more times per day or week, conveniently administered at mealtimes. An effective amount of such a pharmaceutical composition is the amount that provides a clinically significant effect against consumption of fat food. Such amounts will depend, in part, on the particular condition to be treated, age, weight, and general health of the patient, and other factors evident to those skilled in the art. A convenient daily dosage can be in the range from 0.1-4000 mg/kg/day, around 10-1000 mg/kg/day or around 50-500 mg/kg/day. If the body weight of the subject changes during treatment, the dose of the compound might have to be adjusted accordingly.

Any novel feature or combination of features described herein is considered essential to this invention.

The present invention is further illustrated by the following examples, which, however, are not to be construed as limiting the scope of protection. The features disclosed in the foregoing description and in the following examples may, both separately and in any combination thereof, be material for realising the invention in diverse forms thereof.

### EXAMPLES

The snacking model:

Rats are habituated to the presence of a palatable high fat meal (Research diets 45% fat D12451) or another palatable low fat meal (Research diets 10% fat D12450B) in their home cage during four hours in the morning two times a week, 9-13a.m. The rats feed ad libitum on rat chow (Altromin #1324, 4% fat) outside the hours of the session and water is present adlibitum. The consumption of the meal is monitored, and as it stabilises, by 7-9 sessions, the animal is ready for use.

The animals are tested two times a week, and the baseline is re-established within 24h. During the experimental session, the compound is administered 30 minutes before the session starts. Food and water intake is monitored at 2 and 4 hours after the meal is presented.

The following compounds have been tested in the snacking model:

Diazoxide (30 mg/kg PO admin.) reduced the consumption of a high fat meal (45 kcal % fat) with 53% (p<0.01; n=5) and a low fat meal (10 kcal % fat) with 42% (p<0.05; n=5).

6-Chloro-3-isopropylamino-4H-thieno[3,2-e]-1,2,4-thiadiazine 1,1-dioxide (30 mg/kg PO admin.) reduced a high fat meal (45 kcal % fat) consumption with 77% (p<0.01; n=5) and a low fat meal (10 kcal % fat) consumption with 61% (p<0.01; n=5) in the fed male obese Zucker rat.

6-Chloro-3-(1 -methylcyclopropyl)amino-4H-thieno[3,2-e]-1,2,4-thiadiazine 1,1-dioxide (5, 15 and 30 mg/kg PO admin.) reduced a high fat meal (45 kcal % fat) consumption with 44, 64 and 86% respectively (p<0.01; n=5) and a low fat meal (10 kcal % fat) consumption with 15, 57 and 75% (p<0.01; n=5) in the fed male obese Zucker rat.

What is claimed is:

1. A method for reducing the consumption of fat-containing food, said method comprising administering to a subject in need thereof an effective amount of a compound of the general formula (I) 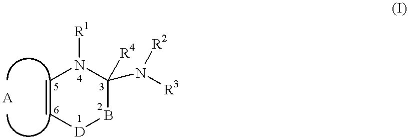
wherein
B represents >NR⁵ or >CR⁵R⁶, wherein R⁵ and R⁶ independently are hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly-substituted with halogen; or R⁵ and R⁴ together represent one of the bonds in a double bond between the atoms 2 and 3 of formula (I);
D represents —S(═O)₂— or —S(═O)—; or
D-B represents —S(═O)(R⁷)═N—
wherein R⁷ is C₁₋₆-alkyl; or aryl or heteroaryl optionally mono- or polysubstituted with halogen, hydroxy, C₁₋₆-alkoxy, aryloxy, arylalkoxy, nitro, amino, C₁₋₆monoalkyl- or dialkylamino, cyano, acyl, or C₁₋₆-alkoxycarbonyl;
R¹ is hydrogen; hydroxy; C₁₋₆-alkoxy; or C₁₋₆-alkyl, C₃₋₆-cycloalkyl, C₂₋₆- alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);
R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen;
R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₋₆-alkyl;
wherein R⁸ is hydrogen; C₃₋₆-cycloalkyl or (C₃₋₆-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;
X is O or S;
R⁹ is hydrogen; C₁₋₆-alkyl; C₂₋₆-alkenyl; C₃₋₆-cycloalkyl optionally mono- or polysubstituted with C₁₋₆-alkyl, halogen, hydroxy or C₁₋₆-alkoxy; or
R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or
R³ is 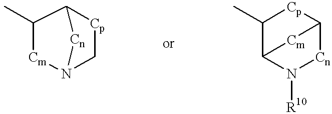
wherein n, m, p independently are 0,1,2,3 and R¹⁰ is hydrogen; hydroxy; C₁₋₆-alkoxy; C₃₋₆-cycloalkyl optionally mono- or poly substituted with C₁₋₆-alkyl, halogen, hydroxy or C₁₋₆-alkoxy; C₁₋₆-alkyl, C₂₋₆-alkenyl or C₂₋₆-alkynyl optionally mono- or polysubstituted with halogen; or
R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;
A together with carbon atoms 5 and 6 of formula (I) represents a 5 or 6 membered heterocyclic system comprising one or more nitrogen-, oxygen- or sulfur atoms, the heterocyclic systems optionally being mono- or poly substituted with halogen; C₁₋₁₂-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl- methyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl- amino; C₁₋₆-monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)-C₁₋₆-alkyl the oxadiazolyl group optionally being substituted with C₁₋₆-alkyl or C₃₋₆-cycloalkyl; or a 5-6 membered nitrogen containing ring, optionally substituted with phenyl or C₁₋₆alkyl; or
a salt thereof with a pharmaceutically acceptable acid or base including all optical isomers of compounds of formula (I), some of which are optically active, and also their mixtures including racemic mixtures, or any tautomeric form thereof.

2. The method according to claim 1 wherein the fat-containing food contains from 10 kcal % fat.

3. The method according to claim 1 wherein the fat-containing food contains at least about 15 kcal % fat.

4. The method according to claim 1 wherein the fat-containing food contains at least about 30 kcal % fat.

5. The method according to claim 1 wherein the fat-containing food contains at least about 45 kcal % fat.

6. The method according to claim 1 wherein the food consumption is related to snack- ing.

7. The method according to claim 1, wherein B is >NR⁵ and R⁵ and R⁴ together represent one of the bonds in a double bond between the atoms 2 and 3 of formula (I).

8. The method according to claim 1, wherein D is —S(═O)₂—.

9. The method according to claim 1, wherein R² is hydrogen or C₁₋₆-alkyl.

10. The method according to claim 1, wherein R³ is R⁸, —OR⁸, NR⁸R⁹ or aryl, the aryl groups optionally being substituted with C₁₋₆-alkyl; wherein
R⁸ is hydrogen; C₃₋₆-cycloalkyl; (C₃₋₆-cycloalkyl)C₁₋₆-alkyl; a 3-6 membered saturated ring system comprising one, two or three nitrogen-, oxygen- or sulfur atoms; or straight or branched C₁₋₁₈-alkyl optionally substituted with halogen, hydroxy, C₁₋₆-alkoxy, C₁₋₆-alkylthio,
C₃₋₆-cycloalkyl or aryl,
R⁹ is hydrogen, C₁₋₆-alkyl or C₃₋₆-cycloalkyl; or
R⁸ and R⁹ together with the nitrogen atom form a 4-6 membered ring.

11. The method according to claim 1, wherein R³ is secondary C₃₋₆-alkyl, tertiary C₄₋₆-alkyl, C₃₋₆-cycloalkyl or (C₃₋₆-cycloalkyl)methyl.

12. The method according to claim 1, wherein A together with carbon atoms 5 and 6 of formula (I) forms a 5 membered heterocyclic system containing one hetero atom selected from nitrogen and sulfur, the heterocyclic system optionally being mono- or disubstituted with halogen; C₁₋₁₂-alkyl; C₃₋₆-cycloalkyl; cyano; cyanomethyl; perhalomethyl; sulfamoyl; C₁₋₆-alkylthio; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl or a 5 - 6 membered nitrogen containing ring, optionally substituted with phenyl or C₁₋₆-alkyl.

13. The method according to claim 1, wherein A together with carbon atoms 5 and 6 of formula (I) forms a 5 membered heterocyclic system containing two hetero atoms selected from nitrogen, oxygen and sulfur, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

14. The method according to claim 1, wherein A together with carbon atoms 5 and 6 of formula (I) forms a 6 membered aromatic heterocyclic system containing one, two or three nitrogen atoms, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylthio; C₁₋₆alkylsulfonyl; C₁₋₆-alkylsulfinyl; arylthio, arylsulfinyl, arylsulfonyl, the aryll group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl: aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5 - 6 membered nitrogen containing ring, optionally substituted with phenyl or C₁₋₆-alkyl.

15. The method according to claim 1, wherein the compound of formula (I) is 6-Chloro-3-(1,2-dimethylpropyl)amino-4H-thieno[3,2-e]-1,2,4-thiadiazine 1,1-dioxide; 6-Chloro-3-ethylamino4H-thieno[3,2-e)-1,2,4-thiadiazine 1,1-dioxide; 6-Chloro-3-isopropylamino-4H-thieno[3,2-e]-1,2,4-thiadiazine 1,1-dioxide; (R)-6-Chloro-3-(p-phenylethyl)amino-4H-thieno[3,2-e]-1 2,4-thiadiazine 1 1-dioxide; 3-Allylamino-6-chloro-4H-thieno[3,2-e]-1,2,4thiadiazine 1,1-dioxide; 6-Chloro-3-cyclopropylamino 4H-thieno[3,2-e]-1,2,4-thiadiazine 1,1-dioxide; 6-Chloro-3-hexylamino -4H-thieno[3,2-e]-1,2,4-thiadiazine 1,1-dioxide; 6-Chloro-3-tetradecylamino-4H-thieno[3,2-e]-1,2,4-thiadiazine 1,1-dioxide; 6-Chloro-3-methylamino-4H-thieno[3,2,e]-1,2,4-thiadiazine 1,1-dioxide; 3-Benzylamino-6-chloro-4H-thieno[3,2,e]-1,2,4-thiadiazine 1,1-dioxide; 6-Chloro-3-octylamino-4H-thieno[3,2-e]-1,2,4-thiadiazine 1,1-dioxide; 6-Chloro-3-isobutylamino-4H-thieno[3,2-e]-1,2,4-thiadiazine 1,1-dioxide; 6-Chloro-3-(4-phenylbutyl)amino-4H-thieno[3,2-e]-1,2,4-thiadiazine 1,1-dioxide; 6-Chloro-3-(1,5-dimethylhexyl)amino-4H-thieno[3,2-e]-1,2,4-thiadiazine 1,1-dioxide; 6-Chloro-3-propylamino-4H-thieno[3,2-e]-1,2,4-thiadiazine 1,1-dioxide; (R)-6-Chloro-3-(2-hydroxy-1-methylethyl)amino-4H-thieno[3,2-e]-1,2,4-thiadiazine 1,1-dioxide; (S)-6-Chloro-3-(2-hydroxy-1-methylethyl)amino-4H-thieno[3,2-e]-1,2,4-thiadiazine 1,1-dioxide; (R)-3-sec-Butylamino-6-chloro-4H-thieno(3,2-e]-1,2,4-thiadiazine 1,1-dioxide; 3-Butylamino-6-chloro-4H-thieno[3,2-e]-1,2,4-thiadiazine 1,1-dioxide; 3-Isopropylamino-7-methyl-4,7-dihydro-pyrazolo[4,3-e][1,2,4]thiadiazine 1,1-dioxide; or
a salt thereof with a pharmaceutically acceptable acid or base including all optical isomers of compounds of formula (I), some of which are optically active, and also their mixtures including racemic mixtures, or any tautomeric form thereof.

16. The method according to claim 1, wherein the compound of formula (I) is 6-Chloro-3-isopropylamino-4H-thieno[3,2-e]-1,2,4-thiadiazine 1,1-dioxide; or
a salt thereof with a pharmaceutically acceptable acid or base including all optical isomers of compounds of formula (I), some of which are optically active, and also their mixtures including racemic mixtures, or any tautomeric form thereof.

17. The method according to claim 1, wherein the compound of formula (I) is 3-Hydrazino-4H-pyrido[4, 3-e]-1,2,4-thiadiazine 1,1-dioxide; 3-Benzylamino-4H-pyrido[4,3-e]-1,2,4-thiadiazine 1,1-dioxide; 3-(R)-(1-Phenylethylamino)-4H-pyrido[4,3-e]-1,2,4-thiadiazine 1,1-dioxide; 3-(S)-(1-Phenylethylamino)-4H-pyrido[4,3-e]-1,2,4-thiadiazine 1,1-dioxide; 3-Benzylamino-7-chloro-4H-pyrido[2,3-e]-1,2,4-thiadiazine 1,1-dioxide; 7-Chloro-3-(R)-(1-phenylethylamino)-4H-pyrido[2,3-e]-1,2,4-thiadiazine 1,1-dioxide; 7-Chloro-3-(S)-(1′-phenylethylamino)-4H-pyrido[2,3-e]-1,2,4-thiadipzine 1,1-dioxide; 3-Benzylamino-4H-pyrido[2,3-e]-1,2,4-thiadiazine 1,1-dioxide; 3-(R)-(1-Phenylethylamino)-4H-pyrido[2,3-e]-1,2,4-thiadiazine 1,1-dioxide; 3-(S)-(1-Phenylethylamino)-4H-pyrido[2,3-e]-1,2,4-thiadiazine 1,1-dioxide; 3-(Hexylamino)4H- pyrido[4,3-e]-1,2,4-thiadiazine 1,1-dioxide; 7-Chloro-3-hexylamino-4H- pyrido[2,3-e]-1,2,4-thiadiazine 1,1-dioxide; 3-Octylamino-4H- pyrido[4,3-e]-1,2,4-thiadiazine 1,1-dioxide; 7-Chloro-3-octylamino-4H- pyrido[2,3-e]-1,2,4-thiadiazine 1,1-dioxide; 3-Allylamino-4H- pyrido[4,3-e]-1,2,4-thiadiazine 1,1-dioxide; 3-Allylamino-7-chloro-4H-pyrido[2,3-e]-1,2,4-thiadiazine 1,1-dioxide; 7-Chloro-3-(2-methoxy-1-methylethyl)amino-4H-pyrido[2,3-e]-i,2,4-thiadiazine 1,1-dioxide; 3-(2-Methoxy-1-methylethyl)amino-4H-pyrido[4,3-e]-1,2,4-thiadiazine 1,1-dioxide; 3-(2-Hydroxy-1-methylethyl)amino-4H-pyrido[4,3-e]-1,2,4-thiadiazine 1,1-dioxide; 3-Benzylamino-2-methyl-2H-pyrido[4,3-e]1,2,4-thiadiazine 1,1-dioxide; 2-Isopropylamino-3,3-dimethoxy-3H-pyrido[2,3-b][1,4]thiazine 4,4-dioxide; or
a salt thereof with a pharmaceutically acceptable acid or base including all optical isomers of compounds of formula (I), some of which are optically active, and also their mixtures including racemic mixtures, or any tautomeric form thereof.

18. The method according to claim 1, wherein the compound of formula (I) is 7-Cyano-3-isopropylamino-6-methyl-4H-thieno[2,3-e]-1,2,4-thiadiazine 1,1-dioxide; 7-Cyano-6-methyl-3-propylamino-4H-thieno[2,3-e]-1,2,4-thiadiazine 1,1-dioxide; 6-Chloro-3-isopentylamino-4H-thieno[3,2-e,-1,2,4-thiadiazine 1,1-dioxide; 6-Chloro-3-(1-methylheptyl)amino-4H-thieno[3,2-e]-1,2,4-thiadiazine 1,1-dioxide; 6-Chloro-3-(1-ethylpentyl)amino-4H-thieno[3,2-e-1,2,4-thiadiazine 1,1-dioxide; 6-Chloro-3-(2-methylbutyl)amino-4H-thieno[3,2-e]-1,2,4-thiadiazine 1,1-dioxide; 6-Chloro-3-(2-methylhexyl)amino-4H -thieno[3,2-e]-1,2,4-thiadiazine 1,1-dioxide; 6-Chloro-3-cyclopentylamino-4H-thieno[3,2-e]-1,2,4-thiadiazine 1,1-dioxide; 6-Chloro-3-cyclohexylamino-4H-thieno[3,2-e]-1,2,4-thiadiazine 1,1-dioxide; Ethyl 3-(6-chloro-1,4-dihydro-1,1-dioxothieno[3,2-e]-1λ⁶,2,4-thiadiazin-3-ylamino)-butanoate; 3-(6-Chloro-1,4-dihydro-1,1-dioxothieno[3,2-e]-1λ⁶,2,4-thiadiazin-3-ylamino)butanoic acid; 6-Chloro-3-(3-hydroxy-1-methylpropyl)amino-4H-thieno[3,2-e]-1,2,4-thiadiazine 1,1-dioxide; (R)-6-Chloro-3-(1-phenylethyl)amino-4H-thieno[3,2-e]-1,2,4-thiadiazine 1,1-dioxide; (S)-3-sec-Butylamino-6-chloro-4H-thieno[3,2-e]-1,2,4-thiadiazine 1,1-dioxide; 6-Chloro-3-isopropylamino-4H-thieno[2,3-e]-1,2,4-thiadiazine 1,1-dioxide; 6-Chloro-3-cyclopentylamino-4H-thieno[2,3-e]-1,2,4-thiadiazine 1,1-dioxide; 6-Bromo-3-isopropylamino-4H-thieno[3,2-e]-1,2,4-thiadiazine 1,1-dioxide; 3-Isopropylamino-4H-thieno[3,2-e]-1,2,4-thiadiazine 1,1-dioxide; 6-Fluoro-3-isopropylamino-4H-thieno[3,2-e]-1,2,4-thiadiazine 1,1-dioxide; 3-Cyclobutylamino-5,6-dimethyl-4H-thieno[3,2-e]-1,2,4-thiadiazine 1,1-dioxide; 3-Cyclopentylamino-5,6-dimethyl-4H-thieno[3,2-e]-1,2,4-thiadiazine 1,1-dioxide; 3-Isopropylamino-6,7-dimethyl-4H-thieno[2,3-e]-1,2,4-thiadiazine 1,1-dioxide; 3-Cyclobutylamino-6,7-dimethyl-4H-thieno[2,3-e]-1,2,4-thiadiazine 1,1-dioxide; 3-Cyclopentylamino -6,7-dimethyl-4H-thieno[2,3-e]-1,2,4-thiadiazine 1,1-dioxide; 5-Chloro-3-isopropylamino-4H-thieno[3,2-e]-1,2,4-thiadiazine 1,1-dioxide; 5-Chloro-3-propylamino-4H-thieno[3,2-e]-1,2,4-thiadiazine 1,1-dioxide; 5-Chloro-3-cyclopentylamino4H-thieno[3,2-e]-1,2,4-thiadiazine 1,1-dioxide; 5-Chloro-6-methyl-3-isopropylamino-4H-thieno[3,2-e]-1,2,4-thiadiazine 1,1-dioxide; 6-chloro-3-isopropylamino-5-methyl4H-thieno[3,2-e]-1,2,4-thiadiazine 1,1-dioxide; 6-chloro-3-cyclopentylamino-5-methyl-4H-thieno[3,2-e]-1,2,4-thiadiazine 1,1-dioxide; 6-Fluoro-3-propylamino-4H-thieno[3,2-e]-1,2,4-thiadiazine 1,1-dioxide; 6-Fluoro-3-cyclopentylamino-4H-thieno[3,2-e]-1,2,4-thiadiazine 1,1-dioxide; 5-Fluoro-3-propylamino-4H-thieno[3,2-e]-1,2,4-thiadiazine 1,1-dioxide; 5-Fluoro-3-isopropylamino-4H-thieno[3,2-e]-1,2,4-thiadiazine 1,1-dioxide; 3-Isopropylamino-7-methyl-4H-thieno[2,3-e]-1,2,4-thiadiazine 1,1-dioxide; 6-Chloro-3-cyclobutylamino-4H-thieno[3,2-e]-1,2,4-thiadiazine 1,1-dioxide; 6-Chloro-3-(2-hydroxyethyl)amino-4H-thieno[3,2-e]-1,2,4-thiadiazine 1,1-dioxide; (±)-3-exo-Bicyclo[2.2.1]hept-2-ylamino-6-chloro-4H-thieno[3,2-e]-1,2,4-thiadiazine 1,1-dioxide; (R)-6-Chloro-3-(2-hydroxypropyl)amino-4H-thieno[3,2-e]-1,2,4thiadiazine 1,1-dioxide; 6-Bromo-3-isopropylamino-4H-thieno3,2-e]-1,2,4-thiadiazine 1,1- dioxide; 5,6-Dibromo-3-isopropylamino-4H-thieno[3,2-e]-1,2,4-thiadiazine 1,1-dioxide; 6-Chloro-3-(furan-2-ylmethyl)amino4H-thieno[3,2-e]-1,2,4-thiadiazine 1,1-dioxide; 6-Chloro-3-( -ethylpropyl)amino-4H-thieno[3,2-e]-1,2,4-thiadiazine 1,1-dioxide; 6-Bromo-3-cyclopentylamino-4H-thieno[3,2-e]-1,2,4-thiadiazine 1,1-dioxide; 6-Chloro-3-(2-methylallyl)amino-4H-thieno[3,2e]-1,2,4thiadiazine 1,1-dioxide; 6-Cyano-3-isopropylamino-4H-thieno[3,2-e]-1,2,4-thiadiazine 1,1-dioxide; or
a salt thereof with a pharmaceutically acceptable acid or base including all optical isomers of compounds of formula (I), some of which are optically active, and also their mixtures including racemic mixtures, or any tautomeric form thereof.

19. Th e method according to claim 1, wherein the general formula () is 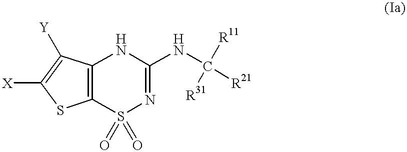
wherein
X and Y independently are hydrogen, halogen, perhalomethyl, C₁₋₆-alkyl or C₁₋₆-alkoxy;
R¹¹, R²¹ and R³¹ independently are C₁₋₆-alkyl, C₂₋₆-alkenyl, C₂₋₆-alkynyl, C₃₋₆-cycloalkyl, carboxy, C₁₋₆-alkoxycarbonyl or aryl, all of which are optionally being mono- or polysubstituted with halogen, hydroxy, oxo, or aryl; or
R¹¹ is as defined above and R²¹—C—R³¹ form a C₃₋₆-cycloalkyl group, optionally being mono- or polysubstituted with C₁₋₆-alkyl, perhalomethyl, halogen, hydroxy or aryl; or
—CR¹¹R²¹R³¹ form a 4- to 12-membered bicyclic or tricyclic carbocyclic system, optionally being mono- or polysubstituted with C₁₋₆-alkyl, perhalomethyl, halogen, hydroxy or aryl; or
a salt thereof with a pharmaceutically acceptable acid or base including all optical isomers of compounds of formula (Ia), some of which are optically active, and also their mixtures including racemic mixtures, or any tautomeric form thereof.

20. The method according to claim 19, wherein X is halogen and Y is hydrogen.

21. The method according to claim 20, wherein X is chloro.

22. The method according to claim 19, wherein R¹¹, R²¹ and R³¹ all are C₁₋₆-alkyl.

23. The method according to claim 19, wherein R¹¹ is methyl.

24. The method according to claim 19, wherein R²¹—C—R³¹ forms a C₃₋₆-cycloalkyl group.

25. The method according to claim 19, wherein —CR¹¹R²¹R³¹ forms a tricyclic carbocyclic system.

26. The method according to claim 19, wherein the compound of formula (Ia) is 3-tert-Butylamino-6-chloro-4H-thieno[3,2-e]-1,2,4-thiadiazine 1,1-dioxide; 6-Chloro-3-(1,1-dimethylpropylamino)-4H-thieno[3,2-e]-1,2,4-thiadiazine 1,1-dioxide; 6-Chloro-3-(1-methylcyclopropyl)amino-4H-thieno[3,2-e]-1,2,4-thiadiazine 1,1-dioxide; 6-Chloro-3-(2-hydroxy-1,1 -dimethylethylamino)-4H-thieno[3,2-e]-1,2,4-thiadiazine 1,1-dioxide; 6-Chloro-3-(1,1,3,3-tetramethylbutylamino)-4H-thieno[3,2-e]-1,2,4-thiadiazine 1,1-dioxide; 3-(1-Adamantyl)amino-6-chloro-4H-thieno[3,2-e]-1,2,4-thiadiazine 1,1-dioxide; 1-(6-Chloro-1,4-dihydro-1,1-dioxo-thieno[3,2-e]-1λ⁶,2,4-thiadiazin-3-ylamino)-cyclopropanecarboxylic acid ethyl ester; 6-Chloro-3-(1-methyl-1-phenylethyl)amino4H-thieno[3,2-e]-1,2,4-thiadiazine 1,1-dioxide; 6-Chloro-3-(1-hydroxymethylcyclopentyl)amino-4H-thieno[3,2-e]-1,2,4-thiadiazine 1,1-dioxide; 1-(6-Chloro-1,4-dihydro-1,1-dioxo-thieno(3,2-e]-1λ⁶,2,4-thiadiazin-3-ylamino)-cyclopropanecarboxylic acid; 6-Chloro-3-(1-methylcyclobutyl)amino4H-thieno[3,2-e]-1,2,4-thiadiazine 1,1-dioxide; 6-Chloro-3-(1 -methylcyclohexyl)amino-4H-thieno[3,2-e]-1,2,4-thiadiazine 1,1-dioxide; 6-Chloro-3-(1-methylcyclopentyl)amino-4H-thieno[3,2-e]-1,2,4-thiadiazine 1,1-dioxide; 6-Chloro-3-(1-ethylcyclobutyl)amino-4H-thieno[3,2-e]-1,2,4-thiadiazine 1,1-dioxide; or
a salt thereof with a pharmaceutically acceptable acid or base including all optical isomers of compounds of formula (Ia), some of which are optically active, and also their mixtures including racemic mixtures, or any tautomeric form thereof.

27. The method according to claim 19, wherein the compound of formula (Ia) is 6-Chloro-3-(1-methylcyclopropyl)amino-4H-thieno[3,2-e]-1,2,4-thiadiazine 1,1-dioxide, or a salt thereof with a pharmaceutically acceptable acid or base including all optical isomers of compounds of formula (Ia), some of which are optically active, and also their mixtures including racemic mixtures, or any tautomeric form thereof.

28. A method for reducing the consumption of fat-containing food, said method comprising administering to a subject in need thereof an effective amount of a potassium channel agonist.

29. The method according to claim 28 wherein the fat-containing food contains at least about 10 kcal % fat.

30. The method according to claim 28 wherein the fat-containing food contains at least about 15 kcal % fat.

31. The method according to claim 28 wherein the fat-containing food contains at least about 30 kcal % fat.

32. The method according to claim 28 wherein the fat-containing food contains at least about 45 kcal % fat.

33. The method according to claim 28 wherein the food consumption is related to snacking.

34. The method according to claim 28 wherein the potassium channel agonist is diazoxide.

35. The method according to claim 28 wherein the potassium channel agonist is a β-cell selective potassium channel agonist.

36. The method according to claim 1 wherein the compound is present in a pharmaceutical composition in a form suitable for oral administration.

37. A method for reducing or lowering the consumption of fat food comprising administering to a subject in need thereof an effective amount of a compound of formula (I) or (Ia) defined in anyone of the preceding claims 1-27, or a pharmaceutically acceptable salt thereof.

38. A method for reducing or lowering the consumption of fat food comprising administering to a subject in need thereof an effective amount of a potassium channel agonist defined in anyone of the preceding claims 28-35, or a pharmaceutically acceptable salt thereof.

39. A method according to any of the claims 37-38 wherein the fat food contains from 10 kcal % fat.

40. A method according to any of the claims 37-38 wherein the fat food contains from 15 kcal % fat.

41. A method according to any of the claims 37-38 wherein the fat food contains from 30 kcal % fat.

42. A method according to any of the claims 37-38 wherein the fat food contains from 45 kcal % fat.

43. A method according to any of the claims 37-38 wherein the fat food consumption is related to snacking.
